(19) United States
(12) Patent Application Publication
(10) Pub. No.: US 2021/0307876 A1
(43) Pub. Date: Oct. 7, 2021

(54) HAND TOOL WITH MOLAR AS RESISTANCE POINT

(71) Applicants: CHIEH-MAO LIN, Taoyuan (TW); CHIH-CHUNG HU, Taoyuan (TW); YI-CHIAN WANG, Taoyuan (TW)

(72) Inventors: CHIEH-MAO LIN, Taoyuan (TW); CHIH-CHUNG HU, Taoyuan (TW); YI-CHIAN WANG, Taoyuan (TW)

(21) Appl. No.: 16/842,451

(22) Filed: Apr. 7, 2020

(51) Int. Cl. A61C 5/00

(52) U.S. Cl. CPC A61C 5/007; A63B 23/032

(57) ABSTRACT

An improved hand tool with molar as resistance point comprises a holding unit, a pivoting unit, an expansion unit and a clean jacket. The holding unit has a first end and a second end. The first end is provided for holding by a user, and the pivoting unit is pivotally connected to the second end correspondingly. A butt end and a connection end are respectively formed at two ends of the expansion unit, the butt end correspondingly butts a plurality of molars of the user, the connection end is pivotally connected to the pivoting unit correspondingly, and the clean jacket is correspondingly sleeved on the expansion unit.

### BACKGROUND OF THE INVENTION

### Field of Invention

This present invention relates to an oral cavity expansion tool, and more particularly to an improved hand tool with molar as resistance point.

### Related Art

For patients with restricted oral cavity opening angle, an oral cavity expansion device is usually required to perform oral cavity opening exercise, oral cavity cleaning, or medical operations in their oral cavities.

The conventional oral cavity expansion devices generally use the upper and lower rows of incisors and canines in an oral cavity as resistance points. Therefore, in the process of opening an oral cavity, the incisors and canines will bear a considerable force. If it is not handled properly, the incisors or canines may be damaged.

In addition, since the conventional oral cavity expansion devices directly reach into the patient's oral cavity and directly butt the molars, this will cause a feeling of friction between the patient's molars and the oral cavity expansion device, which will cause discomfort, and hygiene problems have to be considered also.

In addition, the conventional oral cavity expansion devices do not have any scale or a structure that allows the patient to record a scale suitable for himself. Therefore, when the patient uses it next time, he must spend time again to re-measure the optimal expansion angle, which is very inconvenient.

Therefore, how to solve the above-mentioned problems and drawbacks in the prior art is what the inventor of the present invention and relevant manufacturers engaged in this industry are eager to research and make improvement.

### SUMMARY OF THE INVENTION

Therefore, in order to effectively solve the above problems, a main object of the present invention is to provide an improved hand tool with molar as resistance point that greatly improves the comfort and cleanliness of use.

A secondary object of the present invention is to provide an improved hand tool with molar as resistance point capable of having a required angle adjusted by a user according to requirement.

A secondary object of the present invention is to provide an improved hand tool with molar as resistance point that greatly improves convenience of use.

In order to achieve the above objects, the present invention provides an improved hand tool with molar as resistance point comprising a holding unit, a pivoting unit, an expansion unit and a clean jacket. The holding unit has a first end and a second end. The first end is provided for holding by a user, and the pivoting unit is pivotally connected to the second end correspondingly. A butt end and a connection end are respectively formed at two ends of the expansion unit, the butt end correspondingly butts a plurality of molars of the user, the connection end is pivotally connected to the pivoting unit correspondingly, and the clean jacket is correspondingly sleeved on the expansion unit.

Through the design of the structure of the present invention, the clean jacket is correspondingly sleeved on the expansion unit, so that the discomfort and hygiene problems caused by the user directly butting the butt end of the expansion unit on the molars can be improved, thereby greatly enhancing the comfort and cleanliness of use.

In addition, since the holding unit and the expansion unit are configurations at a same side opposite to the pivoting unit, the user can hold the holding unit, insert the expansion unit into an oral cavity, and then after butting the butt end against the molars correspondingly, a first hold portion and a second hold portion of the holding unit are operated to separate a first expansion arm and a second expansion arm of the expansion unit, thereby expanding the oral cavity and achieving the efficacy of self-operation.

Furthermore, the improved hand tool further has an expansion angle measurement unit. A surface of the expansion angle measurement unit is formed with a plurality of scales, the scales are provided for the user to adjust to an appropriate scale to achieve the most comfortable state when opening the mouth, and to record its scale.

### BRIEF DESCRIPTION OF THE DRAWINGS

FIG. 1A is a perspective exploded view of an improved hand tool with molar as resistance point of the present invention;

FIG. 1B is a partially enlarged view of the improved hand tool with molar as resistance point of the present invention;

FIG. 2 is a schematic diagram of the implementation of the improved hand tool with molar as resistance point of the present invention; and

FIG. 3 is a schematic diagram of the implementation of the improved hand tool with molar as resistance point of the present invention.

### DETAILED DESCRIPTION OF THE INVENTION

The above objects of the present invention, as well as its structural and functional features, will be described in accordance with the preferred embodiments of the drawings.

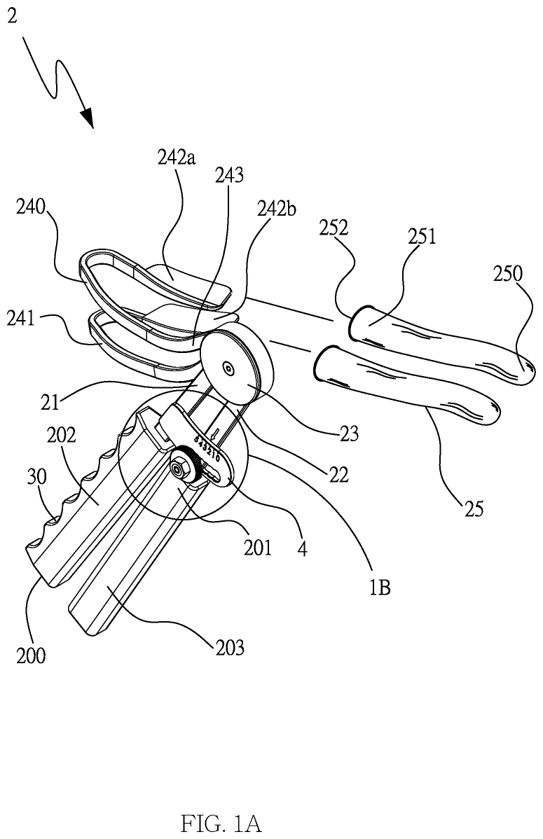

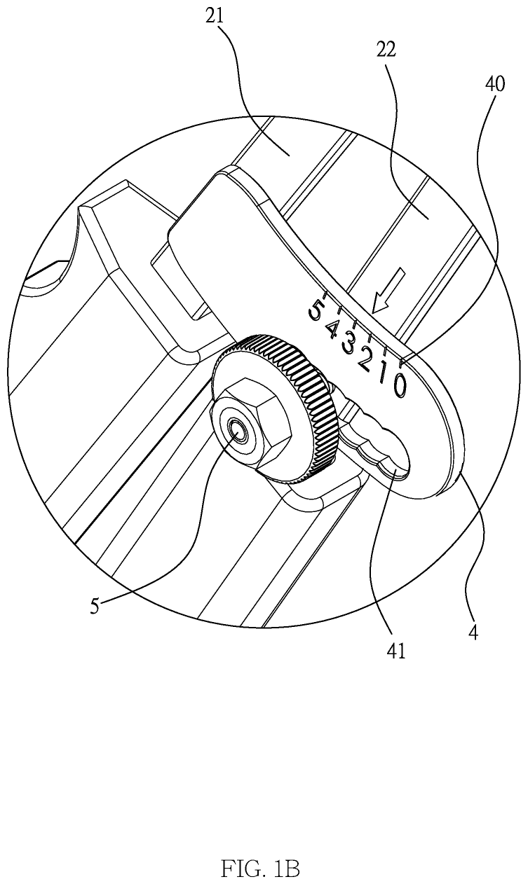

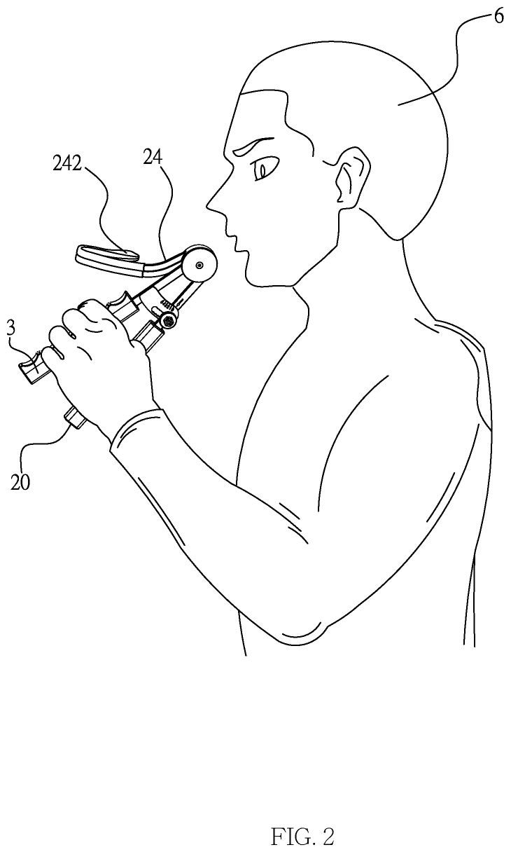

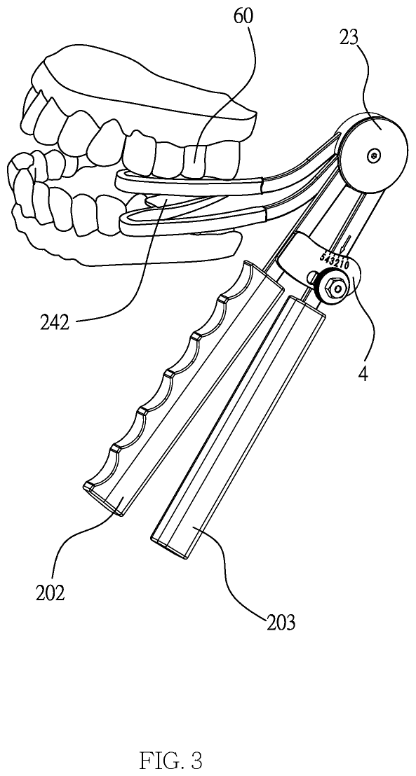

Please refer to FIGS. 1A to 3, which are drawings for an improved hand tool with molar as resistance point of the present invention. As shown in the figures, an improved hand tool with molar as resistance point 2 comprises a holding unit 20, a pivoting unit 23, an expansion unit 24 and a clean jacket 25. The holding unit 20 further has a first hold portion 202 and a second hold portion 203, the first hold portion 202 and the second hold portion 203 have a first end 200 and a second end 201, respectively. The first end 200 is provided for a user 6 to hold and operate conveniently. In addition, the first hold portion 202 and the second hold portion 203 of the holding unit 20 are respectively further sleeved with a grip 3 correspondingly, and the grip 3 is formed with a plurality of grooves 30 corresponding to arc shapes of the fingers of the user 6 for the user 6 to hold firmly and comfortably.

The pivoting unit 23 is pivotally connected to the second end 201 of the holding unit 20 correspondingly. To further explain, a first connecting member 21 is further provided between the pivoting unit 23 and the first hold portion 202, a second connecting member 22 is further provided between the pivoting unit 23 and the second hold portion 203, and the first connecting member 21 and the second connecting member 22 are pivotally corresponding to the pivoting unit 23 to drive the first hold portion 202 and the second hold portion 203.

A butt end 242 and a connection end 243 are respectively formed at two ends of the expansion unit 24. To further explain, the expansion unit 24 further has a first expansion arm 240 and a second expansion arm 241. The butt end 242 further has a first butt portion 242a and a second butt portion 242b. The first expansion arm 240 is connected to the first butt portion 242a, the second expansion arm 241 is connected to the second butt portion 242b, and the first expansion arm 240 and the second expansion arm 241 are correspondingly opened or closed. The first butt portion 242a and the second butt portion 242b correspondingly butt a plurality of upper molars 60 and a plurality of lower molars 60 of the user 6 respectively, and the expansion unit 24 is pivotally connected to the pivoting unit 23 through the connection end 243.

The clean jacket 25 further forms a closed end 250 and an opened end 251 which collectively define a sleeve space 252. The clean jacket 25 correspondingly sleeves and covers the expansion unit 24. In detail, the first expansion arm 240 and the second expansion arm 241 as well as the first butt portion 242a and the second butt portion 242b of the expansion unit 24 are correspondingly accommodated in the sleeve space 252 of the clean jacket 25 so that the first expansion arm 240 and the second expansion arm 241 as well as the first butt portion 242a and the second butt portion 242b are completely covered by the clean jacket 25, and a material of the clean jacket 25 can be made of plastic or other high polymer materials.

Another technical feature of the present invention is that the holding unit 20 and the expansion unit 24 are configurations at a same side opposite to the pivoting unit 23; and therefore the user 6 can hold the first hold portion 202 and the second hold portion 203 of the holding unit 20, insert the expansion unit 24 into an oral cavity, and then after butting the first butt portion 242a and the second butt portion 242b against the upper molars 60 and the lower molars 60 of the user 6 correspondingly, the first hold portion 202 and the second hold portion 203 of the holding unit 20 are operated to separate the first expansion arm 240 and the second expansion arm 241 of the expansion unit 24. Thereby expanding the oral cavity and achieving the efficacy of self-operation.

In addition, an expansion angle measurement unit 4 is further installed between the first connecting member 21 and the second connecting member 22 of the hand tool 2 of the present invention, a surface of the expansion angle measurement unit 4 is formed with a plurality of scales 40, and a through hole 41 is further formed below each of the scales 40 correspondingly. Therefore, the user 6 can adjust to an optimal expansion angle by himself when using the hand tool 2, and then lock the expansion angle measurement unit 4 by using a fixing member 5 to insert into the through hole 41 correspondingly. In addition to maintaining and fixing the expansion angle required by the user 6, the scale 40 on the expansion angle measurement unit 4 can also be referenced and recorded for subsequent use.

Therefore, through the design of the structure of the present invention, the clean jacket 25 is correspondingly sleeved and covered on the first expansion arm 240 and the second expansion arm 241 as well as the first butt portion 242a and the second butt portion 242b, so that the discomfort and hygiene problems caused by the user 6 directly butting the first butt portion 242a and the second butt portion 242b of the expansion unit 24 on the molars 60 can be improved, thereby greatly enhancing the comfort and cleanliness of use.

In addition, it should be explained that the implementation of the present invention is illustrated with the hand tool 2 correspondingly butted in the left oral cavity of the user 6. If the user 6 needs to expand the right oral cavity; similarly, in the same manner, the butt end 242 of the hand tool 2 is butted against the molars 60 in the right half side of the oral cavity, and then the first hold portion 202 and the second hold portion 203 of the holding unit 20 are operated to separate the first expansion arm 240 and the second expansion arm 241 of the expansion unit 24, thereby expanding the oral cavity.

As mentioned above, compared with the prior art, the present invention has the following advantages:

1. Significantly improve the comfort and cleanliness of use;

2. Required angle can be adjusted by the user according to requirement;

3. Greatly improve the convenience of use.

It is to be understood that the above description is only preferred embodiments of the present invention and is not used to limit the present invention, and changes in accordance with the concepts of the present invention may be made without departing from the spirit of the present invention, for example, the equivalent effects produced by various transformations, variations, modifications and applications made to the configurations or arrangements shall still fall within the scope covered by the appended claims of the present invention.

What is claimed is:

1. An improved hand tool with molar as resistance point comprising:
a holding unit having a first end and a second end, the first end being provided for holding by a user;
a pivoting unit pivotally connected to the second end correspondingly;
an expansion unit, a butt end and a connection end being respectively formed at two ends of the expansion unit, the butt end correspondingly butting a plurality of molars of the user, the connection end being pivotally connected to the pivoting unit correspondingly; and
a clean jacket correspondingly sleeved on the expansion unit.

2. The improved hand tool with molar as resistance point as claimed in claim 1, wherein the holding unit and the expansion unit are disposed at a same side opposite to the pivoting unit.

3. The improved hand tool with molar as resistance point as claimed in claim 1, wherein the holding unit is further sleeved with a grip correspondingly, and the grip is formed with a plurality of grooves correspondingly for the user to hold firmly.

4. The improved hand tool with molar as resistance point as claimed in claim 1, wherein the expansion unit further has a first expansion arm and a second expansion arm, the butt end further has a first butt portion and a second butt portion, the first expansion arm is connected to the first butt portion, the second expansion arm is connected to the second butt portion, and the first expansion arm and the second expansion arm are correspondingly opened or closed.

5. The improved hand tool with molar as resistance point as claimed in claim 4, wherein the clean jacket further forms a closed end and an opened end which collectively define a sleeve space, the first expansion arm and the second expansion arm as well as the first butt portion and the second butt portion are correspondingly accommodated in the sleeve space.

6. The improved hand tool with molar as resistance point as claimed in claim 1, wherein the holding unit further has a first hold portion and a second hold portion, two ends of a first connecting member are respectively connected to the first hold portion and the pivoting unit, and two ends of a second connecting member are respectively connected to the second hold portion and the pivoting unit.

7. The improved hand tool with molar as resistance point as claimed in claim 6, wherein an expansion angle measurement unit is further installed between the first connecting member and the second connecting member, and a surface of the expansion angle measurement unit is formed with a plurality of scales.

8. The improved hand tool with molar as resistance point as claimed in claim 7, wherein a through hole is further formed below each of the scales correspondingly, and a fixing member is inserted into the through hole correspondingly.

9. The improved hand tool with molar as resistance point as claimed in claim 1, wherein the clean jacket is made of plastic or other high polymer materials.
